(19) United States
(12) Patent Application Publication
(10) Pub. No.: US 2021/0174900 A1
(43) Pub. Date: Jun. 10, 2021

(54) Methods for Introducing Mutations That Alter the Probability of Intranucleic Acid Base Pairing of a Conserved Structured Nucleotide and Related Compositions

(71) Applicant: CureLab, Inc., Dedham, MA (US)

(72) Inventors: Andrey Chursov, Dedham, MA (US); Alexander Shneider, Dedham, MA (US)

(21) Appl. No.: 17/075,690

(22) Filed: Oct. 20, 2020

Related U.S. Application Data
(63) Continuation of application No. 15/121,560, filed on Aug. 25, 2016.
(60) Provisional application No. 61/945,766, filed on Feb. 27, 2014.

(51) Int. Cl. G16B 30/00; G16B 20/00; A61K 39/145; C12N 7/00; G06F 17/18

(52) U.S. Cl. CPC G16B 30/00; G16B 20/00; A61K 2039/5254; C12N 7/00; G06F 17/18; A61K 39/145

(57) ABSTRACT

The present invention relates generally to methods for introducing mutations that alter the probability of intranucleic acid base pairing of a conserved structured nucleotide in a nucleic acid. The present invention also provide methods for making mutant pathogenic organisms suitable as live attenuated vaccines, animal and human diagnostics, and for identifying suitable drug targets.

### RELATED APPLICATIONS

This application claims the benefit of priority to U.S. Provisional patent Application Ser. No. 61/945,766, filed Feb. 27, 2014 and entitled “Methods for Introducing Mutations that Alter the Probability of Intranucleic Acid Base Pairing of a Conserved Structured Nucleotide and Related Composition,” naming Andrey Chursov and Alexander Shneider as inventors; and designated Attorney Docket No. 151-00103.PRV. The entire content of the foregoing provisional application is incorporated herein by reference, including all text, tables and drawings.

### FIELD

The disclosure relates generally to methods of identifying suitable nucleotide regions for introducing a mutation and related uses.

### BACKGROUND

RNA structure is important for the function and regulation of RNA and it plays a key role in many biological processes. For example, tRNA structure is critical to its proper function in being recognized by the cognate tRNA synthetase and binding to the ribosome and correct mRNA codon. Proper folding of ribosomal RNA (rRNA) is essential to the correct function of the ribosome. Folded structures in viral RNAs have been linked to infectivity, altered splicing, translational frameshifting, packaging, and other functions. In addition, substantial regulation of genes that code for proteins occurs post-transcriptionally, in RNA transport, localization, translation, and degradation. This regulation often occurs through structural elements that affect recognition by specific RNA binding proteins. Thus, the use of folded structures as signals within organisms is not uncommon, nor is it limited to non-protein-encoding RNAs, such as rRNAs, or to non-protein-encoding regions of genomes or messenger RNAs.

The unstable nature of the RNA molecule enables RNA viruses to evolve far more rapidly than DNA viruses, frequently changing their surface structures. RNA viruses in general have very high mutation rates. These mutations of RNA viruses make it more difficult for an organism to develop any kind of lasting immunity to the virus. Mutations occur randomly across the entire length of the viral RNA, and so of course most are not beneficial, producing viruses which lack a needed protein or are otherwise disadvantaged. However, because of the enormous number of offspring produced by each virus, even a high rate of mutation does not threaten the survival of the virus, and when advantageous mutations do occur, they are rapidly selected for and reproduced. This evolution is known as antigenic drift. Thus, at least one reason for the lack of suitable vaccines against most RNA viruses is the high rate of mutability of RNA viruses.

To better understand the biological functions of RNA molecules within a cell and to find out what structural regions can be important for the viral replication and propagation, it is crucial to know their structures. Despite the fact that RNA structures play important roles in different biological processes, the experimental techniques to probe RNA structure are not well developed.

One of the most widely used approaches to analysis of RNA structures (McCaskill J. S., The equilibrium partition function and base pair binding probabilities for RNA secondary structure. Biopolymers, 1990, 29:1105-1119) includes a probabilistic algorithm using the partition function approach that computes base pairing probability and the binding probability for any base. A C program for this algorithm is available in a suite of RNA secondary structure software known as the Vienna RNA package (R. Lorenz, S. H. Bernhart, et al. (2011), “ViennaRNA Package 2.0”, Algorithms for Molecular Biology: 6:26). This package was developed by a theoretical chemistry group at the University of Vienna [Hypertext Transfer Protocol://World Wide Web (dot) tbi (dot) univie (dot) ac (dot) at/RNA/] (Hofacker L. I., et al. Fast folding and comparison of RNA secondary structures. Monatshefte Fr. Chemie. 1994, 125:167-188).

Previous attempts to define “structured” RNA region were confusing them with regions possessing the highest percentage of paired nucleotides. This definition is not satisfactory for two reasons. Firstly, it automatically considers any structure possessing loops (where the nucleotides are not paired) as a “less-structured” element than a double helix only. Secondly, it does not reflect persistence and evolutionary conservation of an RNA structure across different strains.

Effective vaccines, particularly antiviral vaccines, are difficult to make.

Drug and medical diagnostic development are often stymied by a lack of suitable targets for their use.

### SUMMARY

Provided herein are methods to determine regions in RNA polynucleotides which contain evolutionarily conserved secondary structural elements, a method of predicting nucleotide mutations that can be disruptive for such regions, and, in some embodiments, an application of such nucleotide mutations to creating RNA-based vaccines.

Provided herein are methods of introducing a mutation into a nucleic acid that alters the probability of intranucleic acid base pairing of a conserved structured nucleotide by the introduction of a mutation at an identity conserved nucleotide position i, 0<i<Li+1, wherein Li is the length of the nucleic acid sequence, in the nucleotide sequence corresponding to said nucleic acid; determination of the probability of intranucleic acid base pairing for a structure conserved nucleotide position j, 0<j<Lj+1, in said nucleic acid sequence in the presence of the mutation (Pm); comparison of Pm to a threshold probability of intranucleic acid base pairing for a structure conserved nucleotide position j in said nucleic acid sequence by comparison of Pm to Pmin wherein Pmin is a minimum threshold probability of intranucleic acid base pairing for a structure conserved nucleotide position j in said nucleic acid sequence; or, comparison of Pm to Pmax wherein Pmax is a maximum threshold probability of intranucleic acid base pairing for a structure conserved nucleotide position j in said nucleic acid sequence; wherein if Pm<Pmin or Pm>Pmax said mutation is identified as a structure conserved altering mutation; and, introduction of said mutation into said nucleic acid when said mutation is a structure conserved altering mutation. The identity conserved position i can be determined by a method by determination of the probability of a nucleobase occurring at a nucleotide position for each nucleobase, wherein p(A)i, p(U) p(C) i, p(G) i is the probability of adenine, uracil, cytosine or guanine nucleobase occurring at said nucleotide position, respectively; determination of the position-specific mutability Mi at said nucleotide position according to the formula: Mi=−p(A)i*log 2(p(A)i)−p(C) i*log 2(p(C) i)−p(G) i*log 2(p(G) i)−p(U) i*log 2(p(U) i), comparison of Mi to Mmax, wherein Mmax is a maximum threshold mutability; and, determination of said nucleotide position as an identity conserved position when Mi<Mmax. The probability of a nucleobase occurring at a nucleotide position can be determined by determination of the frequency of a nucleobase at a nucleotide position C(B)i among N native variant sequences, wherein B is an adenine, uracil, cytosine or guanine nucleobase; and, determination of said probability of a nucleobase occurring at a nucleotide position by the equation p(B)i=(C(B)i+1)/(N+4). Pmax can be determined by a method where, for a nucleotide position i, 0<i<L+1, where L is the length of said alignment of said native variants of said nucleic acid sequences, determine the mean value of said position-specific set of probabilities. For a nucleotide position i, 0<i<L+1, determine said position-specific range of allowed probabilities as the range from said mean value of said position-specific set of probabilities decreased by said standard deviation of said position-specific set of probabilities multiplied by K, to said mean value of said position-specific set of probabilities increased by said standard deviation of said position-specific set of probabilities multiplied by K. Mmax can be determined by determining mutability values for all nucleotide positions i; and, designating Mmax at a percentile of all mutability values. The percentile can be 1, 2, 2.5, 5, 10, 15, 20, 25, 30, 35, 40, or 50.

In any of the above methods, the structure conserved nucleotide position j can be determined by alignment of N native variant nucleic acid sequences of said nucleic acid where L is the length of the aligned native variant nucleic acid sequences; determination of the probability of intranucleic acid pairing for a nucleotide at position j for each aligned native variant nucleic acid sequence to obtain a plurality of probabilities of intranucleic acid pairing for a nucleotide at position j; determination of the variation (Vj) of said probabilities for said nucleotide at position j; and, comparison of Vj to Vmax, wherein Vmax is a maximum threshold variation of probability; and, determination of said nucleotide position j as a structure conserved nucleotide position when Vj<Vmax. The variation can be standard deviation, standard error or variance. Vmax can be determined where, m for a nucleotide position i, 0<i<L+1, said standard deviation of said position-specific set of probabilities is included into a general set of standard deviations, the mean value and the standard deviation of the values in said general set of standard deviations are calculated, these are the mean standard deviation and the standard deviation of standard deviations, said cutoff value is identified as said standard deviation of standard deviations multiplied by a rational non-negative number and subtracted from said mean standard deviation.

In any of the above methods, the nucleic acid sequence can correspond to mRNA.

In any of the above methods, N can be at least 3.

In any of the above methods, the native variant nucleic acid sequences are non-redundant. The native variant nucleic acid sequences can have identical length.

In any of the above methods, the mutation can be silent.

In any of the above methods, the nucleic acid can be from a gene from a pathogenic organism. The pathogenic organism can be Torque Teno virus (Transfusion transmitted virus), Ippy virus, Lassa fever virus, Lujo virus, Lymphocytic (strains), Lymphocytic choriomeningitis virus (other strains), Mobala virus, Mopcia virus, Amapari virus, Flexal virus, Guanarito virus, Junin virus, Latino virus, Machupo virus, Parana virus, Pichinde virus, Sabia virus, Tamiami virus, Whitewater Arroyo virus, Borna disease virus, Akabane virus, Bhanja virus, Bunyamwera virus, California encephalitis virus, Germiston virus, Oropouche virus, Belgrade (Dobrava) virus, Hantaan virus (Korean haemorrhagic fever), Puumala virus, Prospect Hill virus, Seoul virus, Sin Nombre virus (formerly Muerto Canyon), Crimean/Congo haemorrhagic fever virus, Hazara virus, Rift valley fever virus, Sandfly fever virus, Toscana virus, Norovirus (formerly Norwalk virus), Sapo virus, 29E virus, OC43 virus, SARS virus, Ebola Cote d'Ivoire virus, Ebola Reston virus, Ebola Sudan virus, Ebola Zaire virus, Marburg virus, Absettarov virus, Central European tick-borne encephalitis virus, Dengue viruses types 1-4, GB virus C (Hepatitis G virus), Hanzalova virus, Hepatitis C virus, Hypr virus, Israel turkey meningitis virus, Japanese encephalitis virus, Kumlinge virus, Kyasanur forest disease virus, Louping ill virus, Murray Valley encephalitis virus, Negishi virus, Omsk haemorrhagic fever virus, Powassan virus, Rocio virus, Russian spring summer encephalitis virus, Sal Vieja virus, San Perlita virus, Spondweni virus, St Louis encephalitis virus, Tick-borne encephalitis virus, Wesselsbron virus, West Nile fever virus, Yellow fever virus, Hepatitis B virus, Hepatitis D virus (delta), Cytomegalovirus, Epstein-Barr virus, Herpesvirus simiae (B virus), Herpes simplex virus types 1 and 2, Human herpesvirus type 6—HHV6, Human herpesvirus type 7—HHV7, Human herpesvirus type 8—HHV8 (Kaposi's sarcoma-associated herpesvirus), Varicella-zoster virus, Dhori virus, Influenza virus types A, B and C, Thogoto virus, BK virus, JC virus, KI virus, Simian virus 40 (SV40), WU virus, Human papillomaviruses, Hendra virus (formerly equine morbillivirus), Human metapneumovirus, Measles virus, Mumps virus, Newcastle disease virus, Nipah virus, Parainfluenza virus (Types 1 to 4), Respiratory syncytial virus (human), Bocavirus genus, Parvovirus B19, Human partetravirus (Parv4/Parv5), Acute haemorrhagic conjunctivitis virus (AHC), Coxsackieviruses, Echoviruses, Hepatitis A virus (human enterovirus type 72), Polioviruses, Rhinoviruses, Molluscum contagiosum virus, Buffalopox virus, Cowpox virus, Elephantpox virus, Monkeypox virus, Rabbitpox virus, Vaccinia virus, Variola virus (major and minor), Whitepox virus, Orf virus, Pseudocowpox virus (Milker's nodes virus), Tana virus, Yaba virus, Coltivirus, Human rotaviruses, Orbiviruses, Reoviruses, Human immunodeficiency viruses, Human T-cell lymphotropic viruses (IITLV) types 1 and 2, Simian immunodeficiency virus, Xenotropic murine leukemia virus-related virus, Australian bat lyssavirus, Duvenhage virus, European bat lyssaviruses 1 and 2, Lagos bat virus, Mokola virus, Piry virus, Rabies virus, Vesicular stomatitis virus, Bebaru virus, Chikungunya virus, Eastern equine encephalitis virus, Everglades virus, Getah virus, Mayaro virus, Middleburg virus, Mucambo virus, Ndumu virus, O'nyong-nyong virus, Ross river virus, Sagiyama virus, Semliki forest virus, Sindbis virus, Tonate virus, Venezuelan equine encephalitis virus, Western equine encephalitis virus, Rubella virus, Berne virus, Breda virus, Porcine torovirus, Hepatitis E virus, Actinobacillus actinomycetemcomitans, Actinomadura madurae, Actinomadura pelletieri, Actinomyces gerencseriae, Actinomyces israelii, Actinomyces spp, Alcaligenes spp, Bacillus anthracis, Bacillus cereus, Bacteroides fragilis, Bacteroides spp, Bartonella bacilliformis, Bartonella quintana, Bartonella spp, Bordetella bronchiseptica, Bordetella parapertussis, Bordetella pertussis, Bordetella spp, Borrelia burgdorferi, Borrelia duttonii, Borrelia recurrentis, Borrelia spp, Brachyspira spp (formerly Serpulina spp), Brucella abortus, Brucella canis, Brucella melitensis, Brucella suis, Burkholderia cepacia, Burkholderia mallei (formerly Pseudomonas mallei), Burkholderia pseudomallei (formerly Pseudomonas pseudomallei), Campylobacter fetus, Campylobacter jejuni, Campylobacter spp, Cardiobacterium hominis, Chlamydia trachomatis, Chlamydophila pneumoniae, Chlamydophila psittaci, Clostridium botulinum, Clostridium perfringens, Clostridium tetani, Clostridium spp, Corynebacterium diphtheriae, Corynebacterium haemolyticum, Corynebacterium pseudotuberculosis, Corynebacterium pyogenes, Corynebacterium ulcerans, Corynebacterium spp, Coxiella burnetii, Edwardsiella tarda, Ehrlichia sennetsu (Rickettsia sennetsu), Ehrlichia spp, Eikenella corrodens, Enterobacter aerogenes/cloacae, Elizabethkingia meningoseptica (formerly Flavobacterium meningosepticum), Enterobacter spp, Enterococcus spp, Erysipelothrix rhusiopathiae, Escherichia coli, verocytotoxigenic strains (eg 0157:H7 or O103), Francisella, tularensis (Type A), Francisella, tularensis (Type B), Fusobacterium necrophorum, Fusobacterium spp, Gardnerella vaginalis, Haemophilus ducreyi, Haemophilus influenzae, Haemophilus spp, Helicobacter pylori, Klebsiella oxytoca, Klebsiella pneumoniae, Klebsiella spp, Legionella pneumophila, Legionella spp, Leptospira interrogans (all serovars), Listeria ivanovii, Listeria monocytogenes, Moraxella catarrhalis, Morganella morganii, Mycobacterium africanum, Mycobacterium avium/intracellulare, Mycobacterium bovis, Mycobacterium chelonae, Mycobacterium fortuitum, Mycobacterium kansasii, Mycobacterium leprae, Mycobacterium malmoense, Mycobacterium marinum, Mycobacterium microti, Mycobacterium paratuberculosis, Mycobacterium scrofulaceum, Mycobacterium simiae, Mycobacterium szulgai, Mycobacterium tuberculosis, Mycobacterium ulcerans, Mycobacterium xenopi, Mycoplasma caviae, Mycoplasma hominis, Mycoplasma pneumoniae, Neisseria gonorrhoeae, Neisseria meningitidis, Nocardia asteroides, Nocardia brasiliensis, Nocardia farcinica, Nocardia nova, Nocardia otitidiscaviarum, Pasteurella multocida, Pasteurella spp, Peptostreptococcus anaerobius, Peptostreptococcus spp, Plesiomonas shigelloides, Porphyromonas spp, Prevotella spp, Proteus mirabilis, Proteus penneri, Proteus vulgaris, Providencia alcalifaciens, Providencia rettgeri, Providencia spp, Pseudallescheria boydii, Pseudomonas aeruginosa, Rhodococcus equi, Rickettsia akari, Rickettsia canada, Rickettsia conorii, Rickettsia montana, Rickettsia prowazekii, Rickettsia rickettsii, Rickettsia tsutsugamushi, Rickettsia typhi (Rickettsia mooseri), Rickettsia spp, Salmonella arizonae, Salmonella enterica serovar enteritidis, Salmonella enterica serovar typhimurium 2, Salmonella paratyphi A, Salmonella paratyphi B/java, Salmonella paratyphi C/choleraesuis, Salmonella typhi, Salmonella spp, Shigella boydii, Shigella dysenteriae, Shigella flexneri, Shigella sonnei, Staphylococcus aureus, Streptobacillus moniliformis, Streptococcus agalactiae, Streptococcus dysgalactiae equisimilis, Streptococcus pneumoniae, Streptococcus pyogenes, Streptococcus suis, Streptococcus spp, Treponema carateum, Treponema pallidum, Treponema pertenue, Treponema spp, Ureaplasma parvum, Ureaplasma urealyticum, Vibrio cholerae (including El Tor), Vibrio parahaemolyticus, Vibrio spp, Yersinia enterocolitica, Yersinia pestis, Yersinia pseudotuberculosis, or Yersinia spp

Provided herein are methods for producing a pathogenic organism lacking pathogenicity by determining a mutation according to one of the above for a gene from a pathogenic organism; and, generating a mutant pathogenic organism by introducing a mutation into said pathogenic organism, wherein said mutant pathogenic organism is non-pathogenic.

Provided herein are methods for producing a live attenuated vaccine comprising a pathogenic organism lacking pathogenicity according to claim 17 in a pharmaceutically acceptable preparation. The live attenuated vaccine can include an adjuvant. The adjuvant can be a gel-type, microbial, particulate, oil-emulsion, surfactant-based, or synthetic adjuvant. The live attenuated vaccine can include one or more co-stimulatory components. The co-stimulatory component can be a cell surface protein, a cytokine, a chemokine, or a signaling molecule. The live attenuated vaccine can include one or more molecules that block suppressive or negative regulatory immune mechanisms. The one or more molecules that block suppressive or negative regulatory immune mechanisms can be anti-CTLA-4 antibody, anti-CD25 antibody, anti-CD4 antibody, or IL13Ra2-Fc.

In any of the above methods, the nucleic acid can be a gene from a subject and the gene can be related to a disease or pathogenesis.

Provided herein are methods for identifying human and/or animal mutations which may cause a disease by identification of structured RNA regions for a functionally important gene involved in disease prevention and/or pathogenesis, testing a mutation for its ability to disrupt one or more structured RNA regions of the nucleic acid sequence of said gene. The methods can be used for diagnostic purposes. The methods can be used to identify drug targets.

### BRIEF DESCRIPTION OF THE DRAWINGS

The present invention is illustrated and described herein with reference to the various drawings, in which like reference numbers are used to denote like system components/method steps, as appropriate, and in which:

FIG. 1 depicts standard deviations of probabilities of nucleotides to be in a double-stranded conformation for messenger RNA of NS2 gene of non-pandemic H1N1 influenza A virus. Maximum threshold mutability is depicted with the dashed line;

FIG. 2 depicts standard deviations of probabilities of nucleotides to be in a double-stranded conformation for messenger RNA of M2 gene of non-pandemic H1N1 influenza A virus. Maximum threshold mutability is depicted with the dashed line;

FIG. 3 depicts standard deviations of probabilities of nucleotides to be in a double-stranded conformation for messenger RNA of NS2 gene of pandemic H1N1 influenza A virus. Maximum threshold mutability is depicted with the dashed line;

FIG. 4 depicts standard deviations of probabilities of nucleotides to be in a double-stranded conformation for messenger RNA of M2 gene of pandemic H1N1 influenza A virus. Maximum threshold mutability is depicted with the dashed line;

FIG. 5 depicts moving averages of standard deviations of probabilities of nucleotides to be in a double-stranded conformation for messenger RNA of NS2 gene of non-pandemic H1N1 influenza A virus. Maximum threshold moving average is depicted with the dashed line;

FIG. 6 depicts moving averages of standard deviations of probabilities of nucleotides to be in a double-stranded conformation for messenger RNA of M2 gene of non-pandemic H1N1 influenza A virus. Maximum threshold moving average is depicted with the dashed line;

FIG. 7 depicts moving averages of standard deviations of probabilities of nucleotides to be in a double-stranded conformation for messenger RNA of NS2 gene of pandemic H1N1 influenza A virus. Maximum threshold moving average is depicted with the dashed line;

FIG. 8 depicts moving averages of standard deviations of probabilities of nucleotides to be in a double-stranded conformation for messenger RNA of M2 gene of pandemic H1N1 influenza A virus. Maximum threshold moving average is depicted with the dashed line;

FIG. 9 depicts mutability values (i.e. Shannon entropy) of nucleotide positions for messenger RNA of NS2 gene of non-pandemic H1N1 influenza A virus;

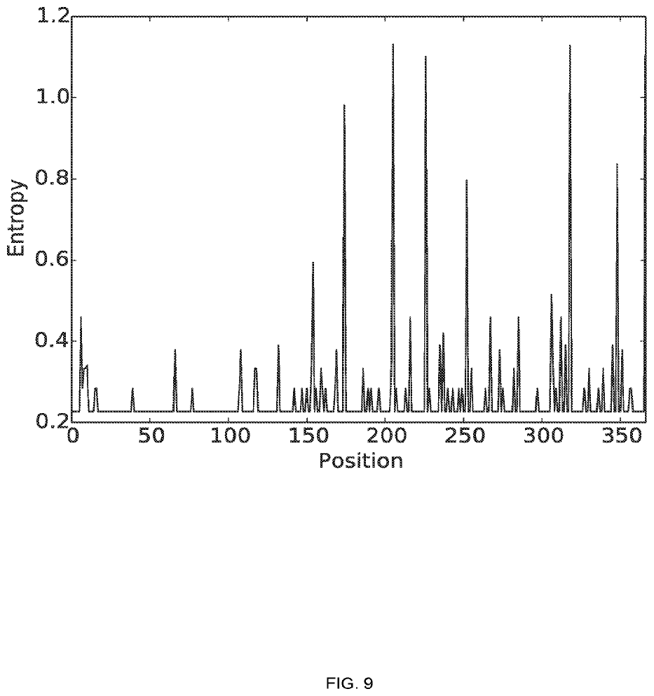

FIG. 10 depicts mutability values (i.e. Shannon entropy) of nucleotide positions for messenger RNA of M2 gene of non-pandemic H1N1 influenza A virus;

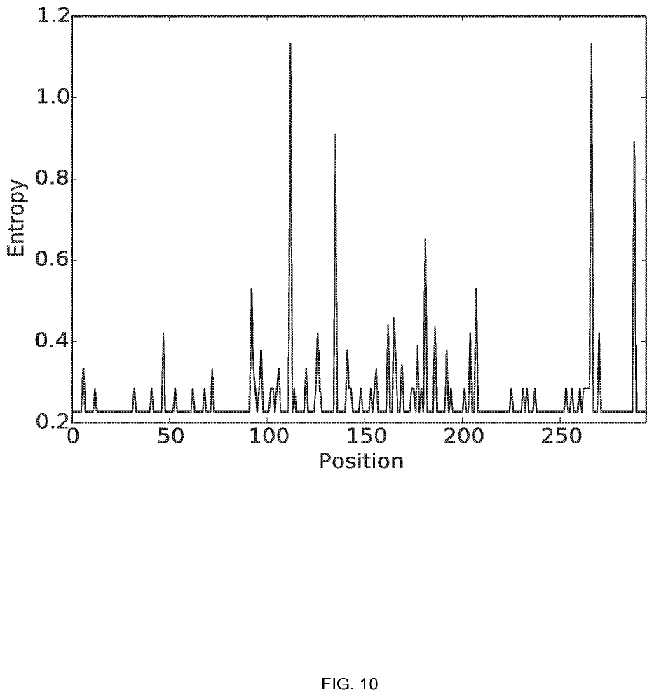

FIG. 11 depicts mutability values (i.e. Shannon entropy) of nucleotide positions for messenger RNA of NS2 gene of pandemic H1N1 influenza A virus; and,

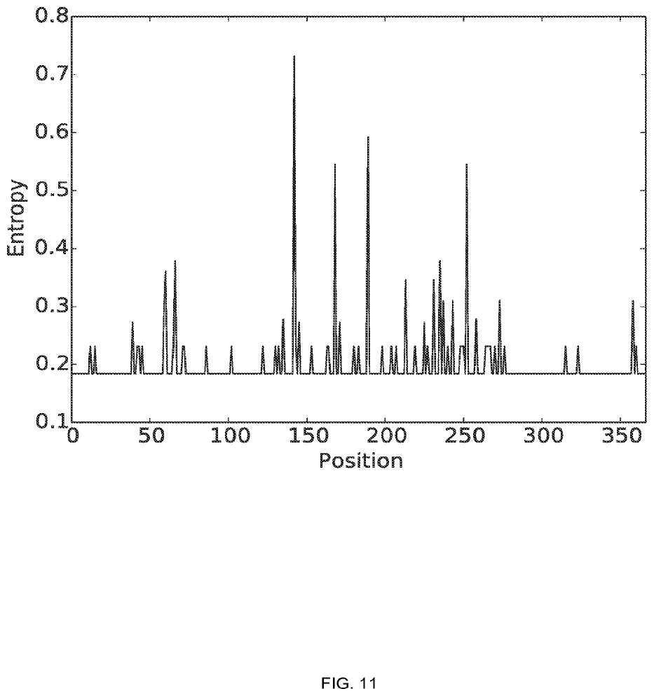

FIG. 12 depicts mutability values (i.e. Shannon entropy) of nucleotide positions for messenger RNA of M2 gene of pandemic H1N1 influenza A virus.

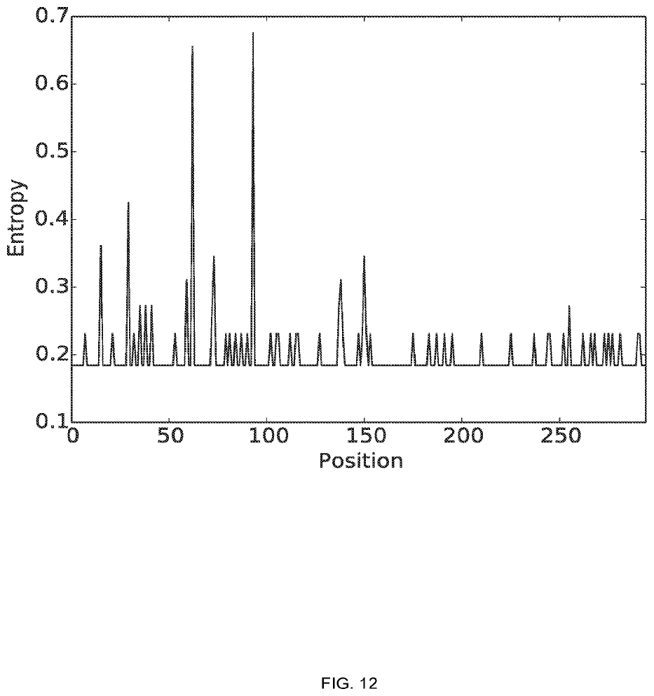

### DETAILED DESCRIPTION

In various exemplary embodiments, provided herein are methods to determine regions in RNA polynucleotides which contain evolutionarily conserved secondary structural elements, a method of predicting nucleotide mutations that can be disruptive for such regions, and, in some embodiments, an application of such nucleotide mutations to creating RNA-based vaccines.

Provided herein is a new definition of structured RNA regions based on alignment of multiple RNA sequences instead of attempting to identify such regions based on analysis of an individual RNA sequence. For example, a stem-loop structure in a particular location that is so important that it is present across the majority of strains means that nucleotides in positions corresponding to the stem would have probabilities to be in a double-stranded conformation close to 1 in all the strains constituting aligned dataset of RNA sequences. At the same time, nucleotides in positions corresponding to the loop would have probabilities to be in a double-stranded conformation close to 0 in all the strains. Thus, structured RNA regions are defined herein as patterns of high and/or low probabilities for the nucleotides to be paired, which manifest across the spectrum of strains.

The methods provided herein answer the long-standing conundrum of why different nucleotides in, for example, the influenza genome mutate with such different frequency. The methods provided herein demonstrate that those nucleotide positions that are the least prone to being mutated do not collocate with regions of conserved RNA structures. Instead, the frequently and/or rarely mutating positions are randomly spread along the RNA sequences. We have demonstrated that in some influenza mRNAs mutations in those nucleotide positions, which are naturally less prone to being mutated, would possess a greater disruptive effect on areas of conserved RNA structures than mutations in positions, which mutate more frequently. As a result, mutations deleterious for vital RNA structures would be eliminated due to the negative selection pressure. This demonstrates that conservation of RNA structures could be a mechanism defining highly differential mutation rate in different influenza nucleotide positions.

Consequently, the methods provided herein enable a new approach for rational design of attenuated vaccines, which allows predicting mutations that would be disruptive for conserved RNA structures. Structurally conserved RNA regions of viral RNAs can be a novel class of anti-viral drug targets. For example, anti-viral agents selectively disrupting RNA structures vital for a viral life cycle identified by the methods provided are useful for anti-viral therapies.

The methods provided herein can be used for rational design of attenuated vaccines. For example, the methods provided can predicting mutation that would be disruptive for structured RNA regions thus making the virus unable to efficient propagation, thereby generating attenuated viral strains, which can be used as vaccines.

As used herein, the term “nucleic acid” refers to strands comprising backbones (e.g., of ribose phosphate and deoxyribose phosphate) and side chains generally comprising heterocyclic bases such as A, C, G, T, and U. Examples of natural nucleic acids include deoxyribonucleic acid (DNA) and ribonucleic acid (RNA).

When referring to a nucleic acid molecule, the term “native” refers to a naturally-occurring (e.g., a wild-type (WT)) nucleic acid.

As used herein, the term “pairing” in reference to nucleotides refers to interaction between nucleotides by the formation of hydrogen bonds. Pairing includes thermodynamically favorable “Watson-Crick” pairs (i.e., G-C and A-U pairs in RNA). Pairing also includes non Watson Crick “mismatch” pairs (G-U pairs in RNA, referred to as “wobble pairs”), which are significantly less stable.

As used herein, the term “primary structure” refers to the sequential order of units in a strand or chain. As used in reference to nucleic acids, the primary structure is the sequence of nucleotides in the nucleic acid strand.

As used herein, the term “secondary structure” refers to the set of the pairing interactions between nucleotides within a single molecule, and can be represented as a list of bases which are paired in a nucleic acid molecule.

As used herein, the term “constraint” refers to an aspect of a structure that might otherwise be variable, but that is assigned a particular value (e.g., a property, position or relationship) during modeling of a structure. Constraints may comprise experimental or theoretically derived aspects of a structure, including but not limited to: distances between components of a structure, (e.g., from NMR NOE measurements or FRET measurements); dihedral angles (e.g., from NMR J-coupling measurements); directions with respect to an axis (e.g., from NMR residual dipolar coupling measurements); exposure of a component to the surface of a structure (as determined by, e.g., EDTA-Fe probing), exposure to solvent (as determined by, e.g., reaction with DMS, DEPC, ENU, CMCT or kethoxal reagents); positions of phosphorus atoms, positions of nucleotides (as determined by, e.g., low resolution X-ray crystallography, cryo-electron microscopy, atomic force microscopy, or NMR methods); other aspects of nucleotide disposition in a structure (e.g., proximity to other nucleotides, paired or unpaired status, or pairing with a particular other nucleotide) such as can be determined by, for example, cross-linking [e.g., using psoralin or mustard reagents) or nuclease sensitivity (e.g., Nucleases Si and V1, or structure-specific nucleases such as FENS).

As used herein, the phrase “sequence identity” means the fraction of identical subunits at corresponding positions in two nucleic acid sequences when the two sequences are aligned to maximize subunit matching, i.e., taking into account gaps and insertions. Sequence alignment can be created using sequence alignment software (e.g., ClustalW, MUSCLE, T-Coffee, etc.).

Methods of the invention compare related variant native nucleic acid sequences. In some embodiments, the variant nucleic acid sequences are from different generations of a particular virus or living organism. The phrases “identity conserved nucleotide position” and “non-mutable nucleotide position” refer to whether a nucleotide position within a nucleic acid will likely have a specific nucleobase (e.g. A, C, G, or U for RNA) at a threshold probability. For example, if at nucleotide position i, wherein 0<i<Li+1 for a nucleic acid of length Li, is found to have an adenine residue above a certain probability threshold among a plurality of native nucleic acids of the same genetic region, then the nucleotide position is scored as an identity conserved position. Every nucleotide position within a nucleic acid can be evaluated as to whether it meets the requirement of an identity conserved position. In an embodiment, the value of Shannon entropy is calculated. The Shannon Entropy H is given by the formula:

$$H = - {\sum }_{i} {p}_{i} {log}_{b} {p}_{i}$$

where pᵢ is the probability of character number i showing up in a stream of characters of the given “script”. In the case of a nucleic acid sequence, such as RNA, the Shannon Entropy is the probability of a given nucleotide appearing in a nucleic acid sequence. Such probabilities, in turn, can be assessed based on the numbers of observed cases of every nucleobase at the particular position in the dataset of native RNA sequences with taking in account pseudocounts. A pseudocount is an amount added to the number of observed cases in order to change the expected probability in a model of those data, when not known to be zero.

Methods of the invention also determine the probability that a nucleotide in a nucleic acid will be paired. The phrases “probability of a nucleotide to be paired”, “probability of a nucleotide to be in a double-stranded conformation”, and “probability of intranucleic acid base pairing” refer to the likelihood that a particular nucleotide position in a nucleic acid molecule is in a paired state with another nucleotide of the same nucleic acid molecule. A “conserved structured nucleotide” refers to whether a nucleotide position within a nucleic acid will likely be paired with another nucleotide within the same nucleic acid at a threshold probability. For example, if at nucleotide position j, wherein 0<(j<Lⱼ+1 for nucleic acid of length Lⱼ, is determined to be in a paired state above a certain probability threshold, the nucleotide position is scored as a structure conserved nucleotide. In an embodiment, the structure conserved nucleotide, whose probability to be paired is significantly less variable than the mean variability of the probabilities of the other ribonucleotides to be paired.

As used herein, the phrase “structure conserved region” refers to a plurality of contiguous nucleotides in a nucleic acid a high density of nucleotides within it that tend to evolutionarily maintain their probability to be in a double-stranded conformation. In contrast to “structure conserved region”, the phrase “non-structured region” refers to a region in RNA polynucleotides with either low density or complete absence of nucleotides within it that tend to evolutionarily maintain their probability to be in a double-stranded conformation.

Provided herein are also methods to determine a suitable mutation in a nucleic acid sequence that alters the probability of intranucleic acid base pairing of a conserved structured nucleotide. For every conserved structured nucleotide position, the native range of probabilities determines the minimum and maximum allowed probability values in such way that it is very likely that the probability of a nucleobase at the particular nucleotide position from every native RNA sequence would be higher than the minimum native probability and lower than the maximum native probability.

Mutations identified by the inventive methods can be introduced into the nucleic acid sequence using standard genetic techniques. The mutation can be a substitution, an insertion or deletion of one or more nucleotides.

In an embodiment, provided herein is a method of introducing a mutation into a nucleic acid that alters the probability of intranucleic acid base pairing of a conserved structured nucleotide that includes (a) the introduction of a mutation at an identity conserved nucleotide position i, 0<i<Li+1, wherein Li is the length of the nucleic acid sequence, in the nucleotide sequence corresponding to said nucleic acid; (b) determination of the probability of intranucleic acid base pairing for a structure conserved nucleotide position j, 0<j<L+1, in said nucleic acid sequence in the presence of the mutation (Pm); (c) comparison of Pm to a threshold probability of intranucleic acid base pairing for a structure conserved nucleotide position j in said nucleic acid sequence by either (i); comparing Pm to Pmin wherein Pmin is a minimum threshold probability of intranucleic acid base pairing for a structure conserved nucleotide position j in said nucleic acid sequence; or, (ii) comparing Pin to Pmax wherein Pmax is a maximum threshold probability of intranucleic acid base pairing for a structure conserved nucleotide position j in said nucleic acid sequence; wherein if Pm<Pmin or Pm>Pmax said mutation is identified as a structure conserved altering mutation; and, (d) introduction of said mutation into said nucleic acid when said mutation is a structure conserved altering mutation.

The method provided herein was used to determine structured regions of H1N1 influenza A strains because of their great public health importance (Spanish flu of 1918, Mexican swine flu, etc.). See Example 1. Additionally, the described method can be easily utilized to find RNA structured regions of other viruses and living organisms.

In an embodiment, a dataset of related RNA sequences is used to determine regions in RNA polynucleotides with high density of nucleotides that tend to evolutionarily maintain their probability to be in a double-stranded conformation.

Quantitative Assessment of a Mutation's Effects on RNA Structuring

The ability of RNA polynucleotides to form particular base pairs and, hence, to form particular RNA secondary structural elements, depends dramatically on the sequence of ribonucleotides in the RNA molecule. Therefore, introducing single nucleotide polymorphism(s) can cause an RNA polynucleotide to become incapable of forming certain RNA secondary structural elements.

In an embodiment, methods of the invention include determining structurally disruptive mutations based on their effect on structured RNA regions (as defined in the previous section).

### EXAMPLES

### Example 1

### Determination of Conserved Structure Nucleotides in H1N1 Influenza a Virus

Sequences of messenger RNAs of H1N1 influenza A virus were analyzed by the method provided herein.

As influenza viruses from different hosts may possess different characteristics, only human influenza strains were utilized in order to eliminate any potential bias. Influenza strains from other hosts (avian, swine, etc.) were excluded from the analysis.

The influenza A genome is composed of eight segments encoding twelve proteins. As two influenza genes, hemagglutinin (HA) and neuraminidase (NA), represent the major viral antigens, these two genes are usually sequenced much more often than any other genes. To eliminate potential bias caused by disproportional representation of similar HA and NA sequences and to make datasets of sequences of different mRNAs comparable to each other, only completely sequenced influenza genomes were used.

Only those strains were selected, which possess identical length for each of their genome segments with other strains in the dataset. The fact that every segment of influenza genome has the same length in every viral genome selected for our work eliminates potential mistakes, which could be introduced by effects of deletion and insertion polymorphisms (DIPs) on the secondary RNA structure. In addition, it automatically ensures that for every mRNA the RNA sequences are aligned without gaps.

Sequences of coding regions of mRNAs of those influenza genomes, which satisfied the above mentioned criteria were downloaded from the Influenza Virus Resource [Hypertext Transfer Protocol://World Wide Web (dot) ncbi (dot) nlm (dot) nih (dot) gov/genomes/FLU/FLU (dot) html].

In order to increase the coherence of the dataset, pandemic influenza strains were separated from non-pandemic influenza strains; thus, two separate datasets were created.

The dataset of RNA sequences should preferably be non-redundant, which means that it should not contain sequences that are characterized by high sequence identity. The level of sequence identity between two RNA sequences in this case can be measured as a fraction of the identical nucleotide positions in a sequence alignment to the total length of the alignment. In other words, to make the datasets non-redundant, only those sequences should be included in the dataset, which have sequence identity levels with every other sequence in the dataset lower than some threshold. The threshold can be any real number in the range of 0 to 1.

Another way of filtering redundant RNA sequences is to exclude RNA sequences, which differ from any other sequence in the dataset by less than some fixed threshold number of nucleotides. In the present analysis, only those influenza strains were included in the datasets, which in the coding regions of their mRNAs have more nucleotide substitutions with coding regions of mRNAs of any other influenza genome from the dataset than 49.

The created datasets of non-pandemic and pandemic influenza A strains consisted of 104 and 135 complete genomes respectively.

RNA Propensity to Form Secondary Structure and Evolutionarily Maintain it and Structured RNA Regions

For each coding region of mRNA sequences from the datasets, the probabilities of nucleotides to be in a double-stranded conformation were calculated with the RNAfold tool from the Vienna RNA Package.

The next step was to identify patterns of nucleotide pairing probabilities, which are repeatedly manifested in the RNAs constituting the dataset. In other words, it is necessary to identify those ribonucleotide positions, whose probability to be paired varies the least from sequence to sequence.

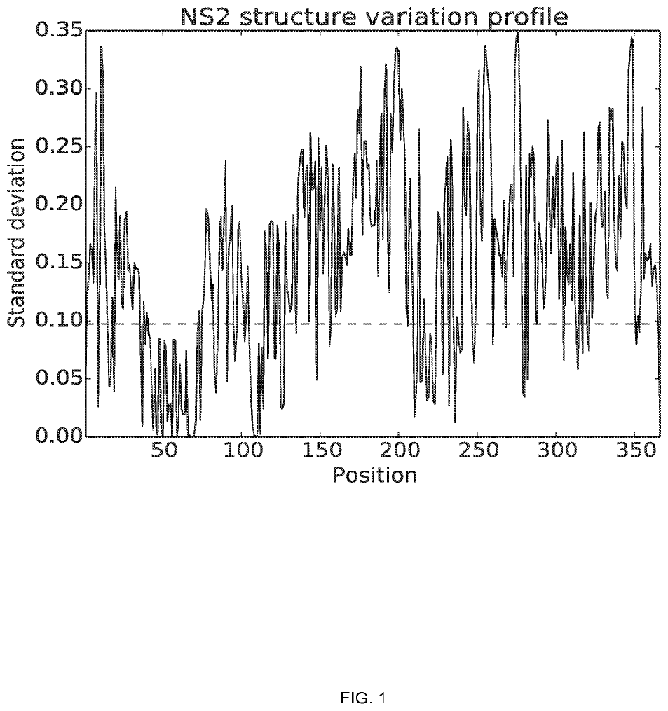

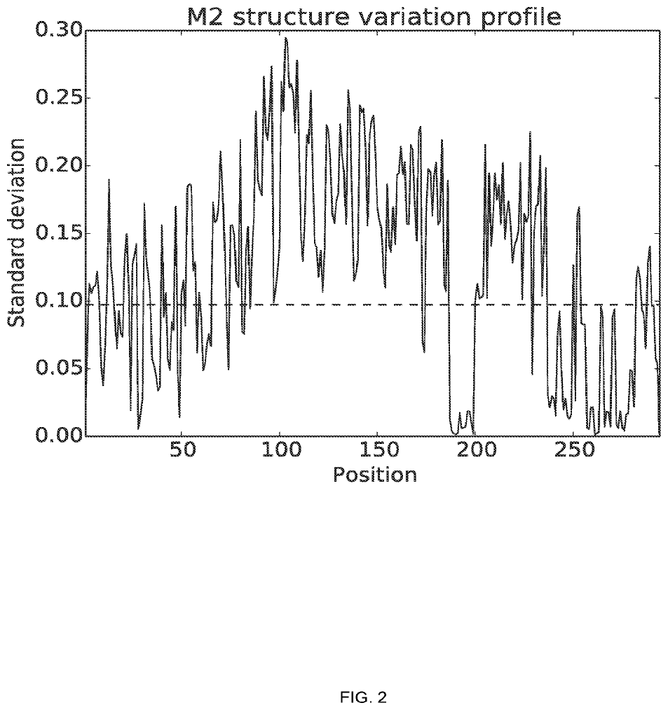

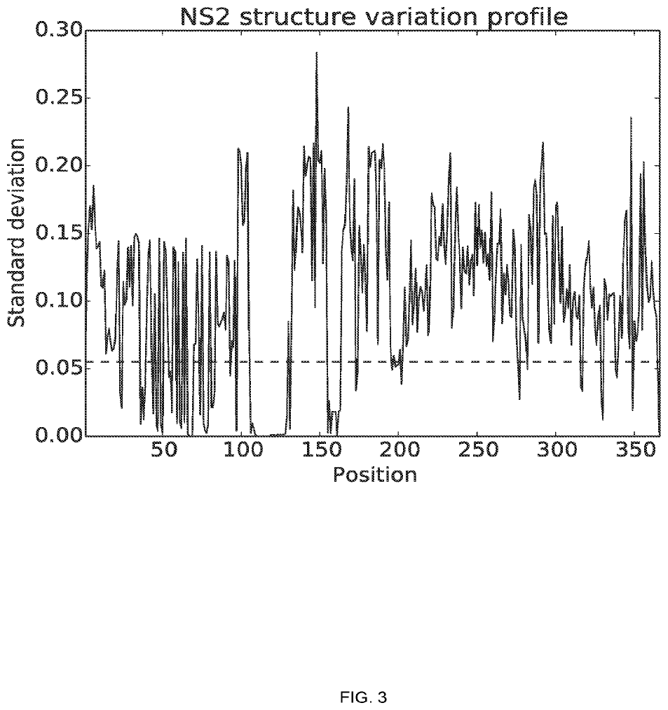

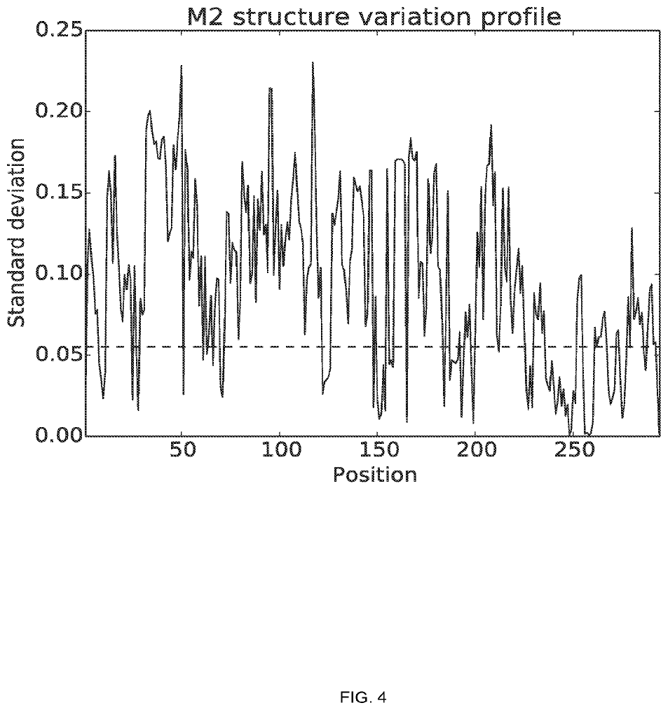

For every ribonucleotide position along the influenza mRNAs, a set of probabilities consisting of 104 and 135 values was computed for the non-pandemic and the pandemic influenza datasets respectively. Standard deviations of these sets of probabilities were calculated for every position. Such standard deviations were used as a measure of structural conservation at a specific nucleotide position (FIGS. 1-4). The novel definition proposed here considers conservation of stems equal to conservation of loops and provides computational friendly quantitative definition of the degree of RNA structure conservation.

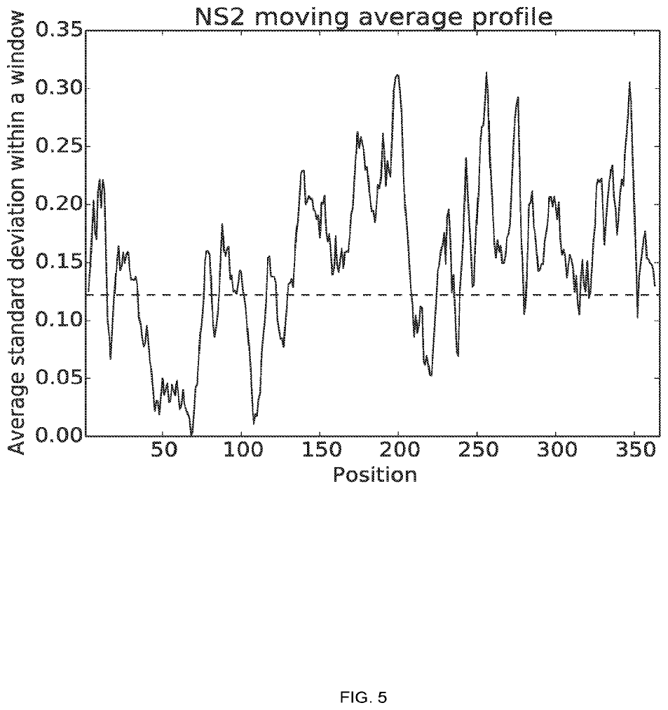

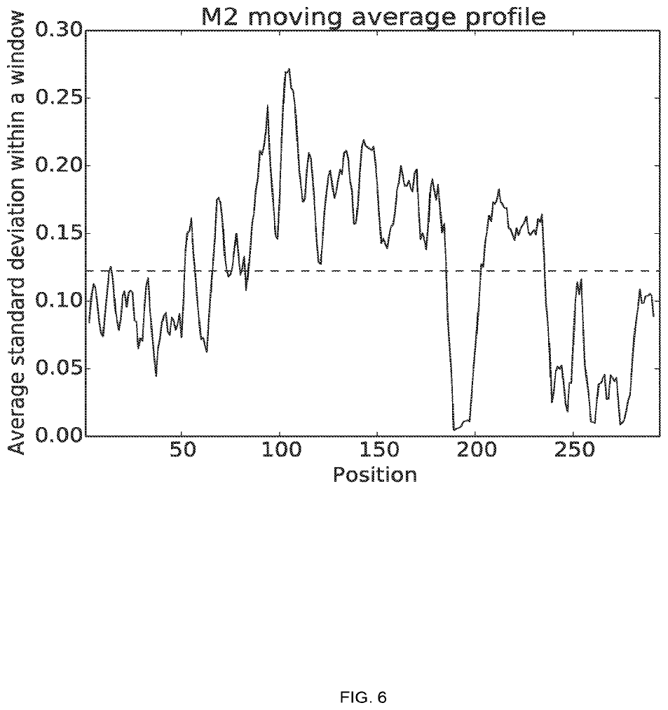

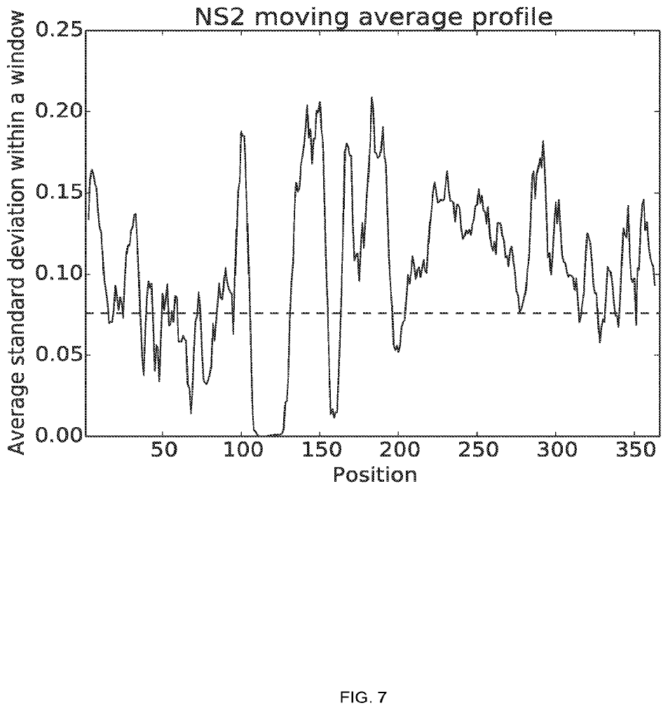

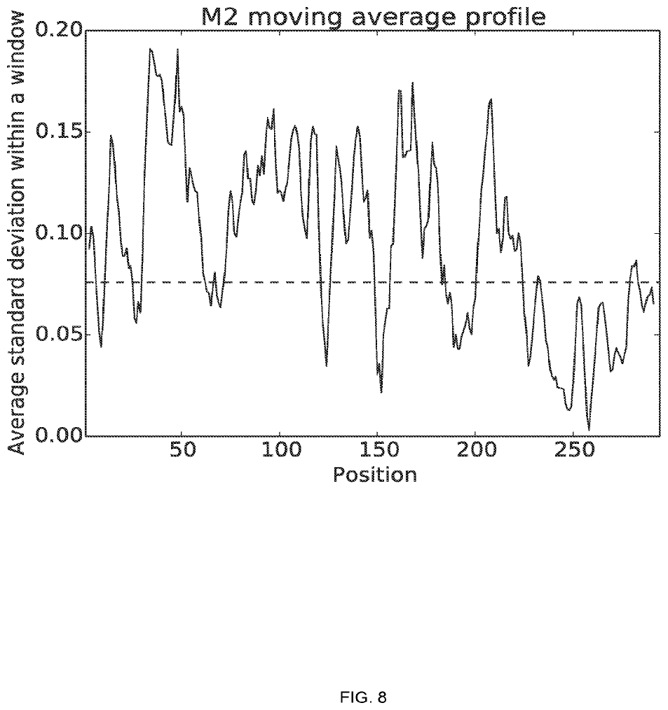

To smooth stochastic fluctuations moving averages of individual standard deviations were calculated for every messenger RNA of influenza virus with a sliding window of size 5 (FIGS. 5-8). Given a series of standard deviation values and a fixed window size, the first element of the moving average is obtained by taking the average of the initial fixed subset of the standard deviation series. The number of values in the initial fixed subset equals the fixed window size. Then the subset is modified by “shifting forward””; that is, excluding the first number of the standard deviation series and including the next number following the original subset in the standard deviation series. This creates a new subset of standard deviation values, which is averaged. This process is repeated over the entire standard deviation series for every coding region of the messenger RNAs of influenza virus. Every moving average value was assigned to the ribonucleotide position, which is in the middle of a corresponding window. The resulting plot line connecting all the computed averages is the moving average.

To determine structured and non-structured regions, all moving average values of individual standard deviations from all influenza mRNAs were combined to one set of moving averages. Mean value and standard deviation of that set of values were calculated. If an individual moving average value of a particular position is less than the overall mean of the moving averages minus the overall standard deviation of the moving averages (this level is depicted with the black dashed line on FIGS. 5-8), this position is considered as “structure conserved”. The combination of structure conserved positions that possess low values of their standard deviations of probabilities of the corresponding ribonucleotides to be in a double-stranded conformation can be defined as structured RNA regions.

### Example 2

### Determination of Structurally Disruptive Mutations in H1N1 Influenza a Virus

As described above, a dataset of aligned influenza sequences was created. For each individual RNA sequence within the dataset, the probability of each nucleotide to be paired was computed. For every nucleotide position within coding regions of influenza mRNA sequences, the mean value and the standard deviation of the probabilities of nucleotides to be paired were calculated. Based on these values, for every nucleotide position within a structured region a range of probabilities from the mean value decreased by the standard deviation to the mean value increased by the standard deviation is considered as naturally occurring range.

Mutations that may potentially disrupt structured RNA regions were in silico randomly introduced into the RNA polynucleotides from the dataset of influenza sequences. The resulting sequences comprise a dataset of random mutants. For every random mutant from the newly generated dataset the probabilities of nucleotides to be in a double-stranded conformation were computed by the RNAfold tool from the Vienna RNA Package.

A mutation occurring in the RNA sequence may have an effect on the probability of each nucleotide within the sequence to be paired. For every random mutant, a number of nucleotides which have their probabilities to be paired that would be outside of a naturally occurring range (as described above, for every particular nucleotide position such range is from a mean value decreased by a standard deviation to a mean value increased by a standard deviation) was computed. If a random mutant has at least one such nucleotide, then mutation(s) that differ the mutant from the original RNA sequence is (are) called disruptive for the structured RNA regions. The effect of a mutation or set of mutations on the RNA structured regions was assessed in a quantitative manner as the number of such nucleotide positions.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. A method of introducing a mutation into a nucleic acid that alters the probability of intranucleic acid base pairing of a conserved structured nucleotide comprising:
a) introduction of a mutation at an identity conserved nucleotide position i, 0<i<L;+1, wherein L; is the length of the nucleic acid sequence, in the nucleotide sequence corresponding to said nucleic acid;
b) determination of the probability of intranucleic acid base pairing for a structure conserved nucleotide position j, 0<j<Lj+1, in said nucleic acid sequence in the presence of the mutation (Pm);
c) comparison of Pm to a threshold probability of intranucleic acid base pairing for a structure conserved nucleotide position j in said nucleic acid sequence comprising;
i) Comparison of Pm to Pmin wherein Pmin is a minimum threshold probability of intranucleic acid base pairing for a structure conserved nucleotide position j in said nucleic acid sequence; or,
ii) Comparison of Pm to Pmax wherein PmaX is a maximum threshold probability of intranucleic acid base pairing for a structure conserved nucleotide position j in said nucleic acid sequence; wherein if Pm<Pmm or Pm>Pmax said mutation is identified as a structure conserved altering mutation; and,
d) introduction of said mutation into said nucleic acid when said mutation is a structure conserved altering mutation.

2. The method of claim 1, wherein said identity conserved position i is determined by a method comprising:
a) determination of the probability of a nucleobase occurring at a nucleotide position for each nucleobase, wherein p(A); p(U) p(C) p(G); is the probability of adenine, uracil, cytosine or guanine nucleobase occurring at said nucleotide position, respectively;
b) determination of the position-specific mutability M; at said nucleotide position according to the formula:
Mi=−p(A)i*log 2(p(A)−p(C);*log 2(p(C);)−p(G);*log 2(p(G);)−p(U);*log 2(p(U);)
c) comparison of Mi to Mmx, wherein Mmx is a maximum threshold mutability; and, d) determination of said nucleotide position as an identity conserved position when M; <Mmx.

3. The method of claim 2, wherein said probability of a nucleobase occurring at a nucleotide position is determined by the method comprising:
a) determination of the frequency of a nucleobase at a nucleotide position C(B)i among N native variant sequences,
b) wherein B is an adenine, uracil, cytosine or guanine nucleobase; and,
c) determination of said probability of a nucleobase occurring at a nucleotide position by the equation p(B); =(C(B+1)/(N+4).

4. The method of claim 1, wherein Pmax is determined by a method comprising:
a) For a nucleotide position i, 0<i<L+1, where L is the length of said alignment of said native variants of said nucleic acid sequences, determine the mean value of said position-specific set of probabilities.
b) For a nucleotide position i, 0<i<L+1, determine said position-specific range of allowed probabilities as the range from said mean value of said position-specific set of probabilities decreased by said standard deviation of said position-specific set of probabilities multiplied by K, to said mean value of said position-specific set of probabilities increased by said standard deviation of said position-specific set of probabilities multiplied by K.

5. The method of claim 2, wherein said Mmax is determined by a method comprising:
a) determine mutability values for all nucleotide positions i; and,
b) designate Mmax at a percentile of all mutability values.

6. The method of claim 5, wherein said percentile is selected from the group consisting of: 1, 2, 2.5, 5, 10, 15, 20, 25, 30, 35, 40, and 50.

7. The method of claim 1, wherein said structure conserved nucleotide position j is determined by a method comprising:
a) alignment of N native variant nucleic acid sequences of said nucleic acid where L is the length of the aligned native variant nucleic acid sequences;
b) determination of the probability of intranucleic acid pairing for a nucleotide at position j for each aligned native variant nucleic acid sequence to obtain a plurality of probabilities of intranucleic acid pairing for a nucleotide at position j;
c) determination of the variation (V,) of said probabilities for said nucleotide at position j; and, d) comparison of Vj to Vmax, wherein Vmax is a maximum threshold variation of probability; and,
e) Determination of said nucleotide position j as a structure conserved nucleotide position when
Claim Vj<Vmax.

8. The method of claim 7, wherein said variation is selected from the group consisting of: standard deviation, standard error and variance.

9. The method of claim 7, wherein Vmax is determined by a method comprising:
a) For a nucleotide position i, 0<i<L+1, said standard deviation of said position-specific set of probabilities is included into a general set of standard deviations.
b) The mean value and the standard deviation of the values in said general set of standard deviations are calculated. These are the mean standard deviation and the standard deviation of standard deviations.
c) Said cutoff value is identified as said standard deviation of standard deviations multiplied by a rational non-negative number and subtracted from said mean standard deviation.

10. The method of claim 1, wherein said nucleic acid sequence corresponds to mRNA.

11. The method of claim 7, wherein N is at least 3.

12. The method of claim 7, wherein said native variant nucleic acid sequences are non-redundant.

13. The method of claim 7, wherein said native variant nucleic acid sequences have identical length.

14. The method of claim 1, wherein said mutation is silent.

15. The method of claim 1, wherein said nucleic acid comprises a gene from a pathogenic organism.

16. The method of claim 18, wherein said pathogenic organism is selected from the group consisting of: Torque Teno virus (Transfusion transmitted virus), Ippy virus, Lassa fever virus, Lujo virus, Lymphocytic (strains), Lymphocytic choriomeningitis virus (other strains), Mobala virus, Mopeia virus, Amapari virus, Flexal virus, Guanarito virus, Junin virus, Latino virus, Machupo virus, Parana virus, Pichinde virus, Sabia virus, Tamiami virus, Whitewater Arroyo virus, Borna disease virus, Akabane virus, Bhanja virus, Bunyamwera virus, California encephalitis virus, Germiston virus, Oropouche virus, Belgrade (Dobrava) virus, Hantaan virus (Korean haemorrhagic fever), Puumala virus, Prospect Hill virus, Seoul virus, Sin Nombre virus (formerly Muerto Canyon), Crimean/Congo haemorrhagic fever virus, Hazara virus, Rift valley fever virus, Sandfly fever virus, Toscana virus, Norovirus (formerly Norwalk virus), Sapo virus, 29E virus, OC43 virus, SARS virus, Ebola Cote d'Ivoire virus, Ebola Reston virus, Ebola Sudan virus, Ebola Zaire virus, Marburg virus, Absettarov virus, Central European tick-borne encephalitis virus, Dengue viruses types 1-4, GB virus C (Hepatitis G virus), Hanzalova virus, Hepatitis C virus, Hypr virus, Israel turkey meningitis virus, Japanese encephalitis virus, Kumlinge virus, Kyasanur forest disease virus, Louping ill virus, Murray Valley encephalitis virus, Negishi virus, Omsk haemorrhagic fever virus, Powassan virus, Rocio virus, Russian spring summer encephalitis virus, Sal Vieja virus, San Perlita virus, Spondweni virus, St Louis encephalitis virus, Tick-borne encephalitis virus, Wesselsbron virus, West Nile fever virus, Yellow fever virus, Hepatitis B virus, Hepatitis D virus (delta), Cytomegalovirus, Epstein-Barr virus, Herpesvirus simiae (B virus), Herpes simplex virus types 1 and 2, Human herpesvirus type 6—HHV6, Human herpesvirus type 7—HHV7, Human herpesvirus type 8—HHV8 (Kaposi's sarcoma-associated herpesvirus), Varicella-zoster virus, Dhori virus, Influenza virus types A, B and C, Thogoto virus, BK virus, JC virus, KI virus, Simian virus 40 (SV40), WU virus, Human papillomaviruses, Hendra virus (formerly equine morbillivirus), Human metapneumo virus, Measles virus, Mumps virus, Newcastle disease virus, Nipah virus, Parainfluenza virus (Types 1 to 4), Respiratory syncytial virus (human), Bocavirus genus, Parvovirus B19, Human partetravirus (Parv4/Parv5), Acute haemorrhagic conjunctivitis virus (AHC), Coxsackieviruses, Echoviruses, Hepatitis A virus (human enterovirus type 72), Polioviruses, Rhinoviruses, Molluscum contagiosum virus, Buffalopox virus, Cowpox virus, Elephantpox virus, Monkeypox virus, Rabbitpox virus, Vaccinia virus, Variola virus (major and minor), Whitepox virus, Orf virus, Pseudocowpox virus (Milker's nodes virus), Tana virus, Yaba virus, Coltivirus, Human rotaviruses, Orbiviruses, Reoviruses, Human immunodeficiency viruses, Human T-cell lymphotropic viruses (HTLV) types 1 and 2, Simian immunodeficiency virus, Xenotropic murine leukemia virus-related virus, Australian bat lyssavirus, Duvenhage virus, European bat lyssaviruses 1 and 2, Lagos bat virus, Mokola virus, Piry virus, Rabies virus, Vesicular stomatitis virus, Bebaru virus, Chikungunya virus, Eastern equine encephalitis virus, Everglades virus, Getah virus, Mayaro virus, Middleburg virus, Mucambo virus, Ndumu virus, O'nyong-nyong virus, Ross river virus, Sagiyama virus, Semliki forest virus, Sindbis virus, Tonate virus, Venezuelan equine encephalitis virus, Western equine encephalitis virus, Rubella virus, Berne virus, Breda virus, Porcine torovirus, Hepatitis E virus, Actinobacillus actinomycetemcomitans, Actinomadura madurae, Actinomadura pelletieri, Actinomyces gerencseriae, Actinomyces israelii, Actinomyces spp, Alcaligenes spp, Bacillus anthracis, Bacillus cereus, Bacteroides fragilis, Bacteroides spp, Bartonella bacilliformis, Bartonella quintana, Bartonella spp, Bordetella bronchiseptica, Bordetella parapertussis, Bordetella pertussis, Bordetella spp, Borrelia burgdorferi, Borrelia duttonii, Borrelia recurrentis, Borrelia spp, Brachyspira spp (formerly Serpulina spp), Brucella abortus, Brucella canis, Brucella melitensis, Brucella suis, Burkholderia cepacia, Burkholderia mallei (formerly Pseudomonas mallei), Burkholderia pseudomallei (formerly Pseudomonas pseudomallei), Campylobacter fetus, Campylobacter jejuni, Campylobacter spp, Cardiobacterium hominis, Chlamydia trachomatis, Chlamydophila pneumoniae, Chlamydophila psittaci, Clostridium botulinum, Clostridium perfringens, Clostridium tetani, Clostridium spp, Corynebacterium diphtheriae, Corynebacterium haemolyticum, Corynebacterium pseudotuberculosis, Corynebacterium pyogenes, Corynebacterium ulcerans, Corynebacterium spp, Coxiella burnetii, Edwardsiella tarda, Ehrlichia sennetsu (Rickettsia sennetsu), Ehrlichia spp, Eikenella corrodens, Enterobacter aerogenes/cloacae, Elizabethkingia meningoseptica (formerly Flavobacterium meningosepticum), Enterobacter spp, Enterococcus spp, Erysipelothrix rhusiopathiae, Escherichia coli, verocytotoxigenic strains (eg 0157:H7 or O103), Francisella tularensis (Type A), Francisella tularensis (Type B), Fusobacterium necrophorum, Fusobacterium spp, Gardnerella vaginalis, Haemophilus ducreyi, Haemophilus influenzae, Haemophilus spp, Helicobacter pylori, Klebsiella oxytoca, Klebsiella pneumoniae, Klebsiella spp, Legionella pneumophila, Legionella spp, Leptospira interrogans (all serovars), Listeria ivanovii, Listeria monocytogenes, Moraxella catarrhalis, Morganella morganii, Mycobacterium africanum, Mycobacterium avium/intracellulare, Mycobacterium bovis, Mycobacterium chelonae, Mycobacterium fortuitum, Mycobacterium kansasii, Mycobacterium leprae, Mycobacterium malmoense, Mycobacterium marinum, Mycobacterium microti, Mycobacterium paratuberculosis, Mycobacterium scrofulaceum, Mycobacterium simiae, Mycobacterium szulgai, Mycobacterium tuberculosis, Mycobacterium ulcerans, Mycobacterium xenopi, Mycoplasma caviae, Mycoplasma hominis, Mycoplasma pneumoniae, Neisseria gonorrhoeae, Neisseria meningitidis, Nocardia asteroides, Nocardia brasiliensis, Nocardia farcinica, Nocardia nova, Nocardia otitidiscaviarum, Pasteurella multocida, Pasteurella spp, Peptostreptococcus anaerobius, Peptostreptococcus spp, Plesiomonas shigelloides, Porphyromonas spp, Prevotella spp, Proteus mirabilis, Proteus penneri, Proteus vulgaris, Providencia alcalifaciens, Providencia rettgeri, Providencia spp, Pseudallescheria boydii, Pseudomonas aeruginosa, Rhodococcus equi, Rickettsia akari, Rickettsia Canada, Rickettsia conorii, Rickettsia montana, Rickettsia prowazekii, Rickettsia rickettsii, Rickettsia tsutsugamushi, Rickettsia typhi (Rickettsia mooseri), Rickettsia spp, Salmonella arizonae, Salmonella enterica serovar enteritidis, Salmonella enterica serovar typhimurium 2, Salmonella paratyphi A, Salmonella paratyphi B/java, Salmonella paratyphi CI choleraesuis, Salmonella typhi, Salmonella spp, Shigella boydii, Shigella dysenteriae, Shigella flexneri, Shigella sonnei, Staphylococcus aureus, Streptobacillus moniliformis, Streptococcus agalactiae, Streptococcus dysgalactiae equisimilis, Streptococcus pneumoniae, Streptococcus pyogenes, Streptococcus suis, Streptococcus spp, Treponema carateum, Treponema pallidum, Treponema pertenue, Treponema spp, Ureaplasma parvum, Ureaplasma urealyticum, Vibrio cholerae (including El Tor), Vibrio parahaemolyticus, Vibrio spp, Yersinia enterocolitica, Yersinia pestis, Yersinia pseudotuberculosis, and Yersinia spp

17. A method for producing a pathogenic organism lacking pathogenicity comprising:
a) Determining a mutation according to the methods of claim 15 or 16 in a gene from a pathogenic organism; and,
b) Generating a mutant pathogenic organism by introducing said mutation into said pathogenic organism, wherein said mutant pathogenic organism is non-pathogenic.

18. A live attenuated vaccine comprising a pathogenic organism lacking pathogenicity according to claim 17 in a pharmaceutically acceptable preparation.

19. The live attenuated vaccine according to claim 18 further comprising an adjuvant.

20. The live attenuated vaccine according to claim 19, wherein said adjuvant is selected from the group consisting of: gel-type, microbial, particulate, oil-emulsion, surfactant-based, and synthetic adjuvant.

21. The live attenuated vaccine according to claim 18, further comprising one or more co-stimulatory components.

22. The live attenuated vaccine according to claim 21, wherein said one or more co-stimulatory components is selected from the group consisting of: a cell surface protein, a cytokine, a chemokine, and a signaling molecule.

23. The live attenuated vaccine according to claim 18, further comprising one or more molecules that block suppressive or negative regulatory immune mechanisms.

24. The live attenuated vaccine according to claim 23, wherein said one or more molecules that block suppressive or negative regulatory immune mechanisms is selected from the group consisting of: anti-CTLA-4 antibody, anti-CD25 antibody, anti-CD4 antibody, and IL13Ra2-Fc.

25. The method of claim 1, wherein said nucleic acid is a gene from a subject and said gene is related to a disease or pathogenesis.

26. A method of identifying human and/or animal mutations which may cause a disease comprising: a) Identification of structured RNA regions for a functionally important gene involved into disease prevention and/or pathogenesis. b) Testing a mutation for its ability to disrupt one or more structured RNA regions of the nucleic acid sequence of said gene.

27. Method of utilizing said mutation identified according to the claim 25 for diagnostic purposes.

28. Method of utilizing said mutation identified according to the claim 25 as drug targets.
